(19) United States
(12) Patent Application Publication
(10) Pub. No.: US 2016/0143638 A1
(43) Pub. Date: May 26, 2016

(54) Adhered Contractional Elastomeric Membrane for Surgical Wound Design and Closure

(71) Applicant: ImplantAdjust LLC, Point Roberts, WA (US)

(72) Inventor: Peter Renke, Vancouver (CA)

(21) Appl. No.: 14/945,259

(22) Filed: Nov. 18, 2015

Related U.S. Application Data
(60) Provisional application No. 62/081,594, filed on Nov. 19, 2014.

(51) Int. Cl. A61B 17/08

(52) U.S. Cl. CPC A61B 17/085; A61B 17/08; A61B 2017/086; A61B 2017/00884

(57) ABSTRACT

A method for surgical wound closure includes providing a pre-stressed elastomeric membrane having an aperture enclosed by at least a portion of the membrane; adhering the pre-stressed membrane to dermal tissue of a patient at a surgical procedure site; creating an incision through dermal tissue of a patient at the surgical procedure site at a location defined at least in part by the aperture of the membrane; and contracting the membrane and adhered dermal tissue, such that the aperture and incision created during the surgical procedure are at least partially closed. A kit of parts for adhered contractional wound closure including an unstressed elastomeric membrane, at least one retaining frame configured to apply a stressing force for pre-stressing the membrane, and an adhesive configured to attach the pre-stressed elastomeric membrane to dermal tissue of a patient, is also disclosed herein.

### CROSS REFERENCE TO RELATED APPLICATIONS

This application claims priority to U.S. Provisional Patent Application No. 62/081,594 filed on Nov. 19, 2014, the disclosure of which is hereby incorporated by reference in its entirety.

### BACKGROUND OF THE INVENTION

1. Field of the Invention

The present disclosure relates generally to a membrane with elastomeric properties that can be temporarily adhered to the epidermis of the skin and, more specifically, to an adhered contractional elastomeric membrane for surgical wound design and closure.

2. Description of the Related Art

Surgical procedures that penetrate the dermal layers to facilitate open access require some form of wound closure. Such wounds are generally closed by suturing and/or stapling for the main closure structure. Taping and tissues adhesives are used to compliment such closures, but may be sufficient in cases of smaller closures.

However, large wound closures present many challenges to surgeons. Surgeons must adapt to the circumstances presented and make decisions based on their assessment of the tissues and available treatment options. All dermatologic wound closures share the common goals to re-establish the necessary protective barrier of the skin and promote healing. The healing process initiates with inflammation, which in turn stimulates the immune system and mobilizes hemostasis. The surgeon must contend with these processes during the procedure. Timely closure of the wound allows for tissue formation and angiogenesis to begin internally and re-epithelialization superficially. The physical wound closure is an important juncture with the reestablishment of the skin barrier. After two weeks, a wound has less than 10% of its strength. The formation of mature collagen structure can take up to a year to develop. When fully healed, a wound area attains a strength equivalent to about 75% of normal uncompromised tissues. A scar will remain that delineates the closure boundaries. Closure strength is essential. The minimizing of scarring and best possible healed appearance is very significant to patient satisfaction.

One of the more common medically necessary wound closures includes diabetic ulcers of the lower leg extremities. Another common procedure is the excision of skin cancers, which results in a wide variety of wounds requiring various closures. Both of these conditions pose challenges for surgeons, which rely on available materials, surgical tools, and the surgeons' skill.

A readily performed elective surgery presenting large wound closure is abdominoplasty. Abdominoplasty is commonly known as a “tummy tuck”. Many women choose to have this procedure done to remove the excessive stretched skin after child bearing. The number of abdominoplasty procedures is reported to have increased by more than 300% since 1997. The popularity and acceptance of these procedures will likely continue to increase with improved results. The presentation of the problem manifests as excess skin and subcutaneous tissue. In many cases, there is also laxity of the abdominal wall musculature. The most common cause of such tissue and muscle laxity is pregnancy, which is compounded by multiple or frequent pregnancies by natural birth or caesarian section. Another indication for abdominoplasty is after massive weight loss. The skin and tissues have inherent limitations as to their ability to stretch and recover. Generally, the level of natural recovery post-partum will be determined by six months with stretch marks, scar tissue, and general laxity having minimal potential to further improve.

Abdominoplasties are performed as either full or partial procedures. A full procedure is generally done when the patient exceeds his or her ideal body weight by more than 20%. In these cases the procedure involves incision around the navel to facilitate extensive tissue removal. The naval is repositioned and a large incision is required along the lower abdomen. The abdominal muscles usually require suturing to tighten them. Liposuction is often performed to achieve more desirable results. A mini tummy tuck is considered a partial abdominoplasty. This less invasive procedure provides great outcomes for relatively fit patients presenting with close to ideal body weights.

### SUMMARY OF THE INVENTION

Generally, the present invention provides methods, arrangements, kits, and devices that utilize the characteristic elasticity of an elastomeric membrane to contract and support involved dermal tissues. Preferred and non-limiting embodiments or aspects of devices, arrangements, kits, and methods disclosed herein are provided in accordance with the objective to improve presently-used wound closure techniques.

Accordingly, and in one preferred and non-limiting aspect or embodiment, provided is a method for surgical wound closure comprising: providing a pre-stressed elastomeric membrane comprising an aperture enclosed by at least a portion of the membrane; adhering the pre-stressed membrane to dermal tissue of a patient at a surgical procedure site; creating an incision through dermal tissue of a patient at the surgical procedure site at a location defined at least in part by the aperture of the membrane; and contracting the membrane and adhered dermal tissue, such that the aperture and incision created during the surgical procedure are at least partially closed.

In one preferred and non-limiting aspect or embodiment, providing the pre-stressed membrane comprises: providing an elastomeric membrane in an unstressed state; and retaining at least a portion of the membrane in a retaining frame, thereby stretching the membrane to a predetermined size and shape to form the pre-stressed membrane. In another preferred and non-limiting aspect or embodiment, contracting the membrane comprises removing the retaining frame to release the membrane to apply a contracting force to the dermal tissue. In a further preferred and non-limiting aspect or embodiment, the elastomeric membrane comprises at least one retaining collar extending from a surface of the membrane, and wherein retaining at least a portion of the membrane in a retaining frame comprises mounting at least one retaining ring to the at least one retaining collar. In a further preferred and non-limiting aspect or embodiment, the elastomeric membrane comprises at least an inner retaining collar and an outer retaining collar, the collars extending from a surface of the membrane, and wherein retaining at least a portion of the membrane in a retaining frame comprises mounting at least one disc-shaped annular retaining shell between the inner retaining collar and the outer retaining collar.

In one preferred and non-limiting aspect or embodiment, the pre-stressed membrane comprises a body and a plurality of tabs extending radially therefrom, the tabs being configured to be adhered to dermal tissue of the patient at varying positions, thereby varying a contracting force provided to different portions of the dermal tissue by the membrane.

In one preferred and non-limiting aspect or embodiment, providing the pre-stressed elastomeric membrane comprises selecting a membrane of a suitable size and shape for a procedure to be performed from a plurality of different sized membranes.

In one preferred and non-limiting aspect or embodiment, providing the pre-stressed elastomeric membrane comprises sizing the membrane by trimming portions of the membrane to produce an elastomeric membrane of an appropriate size and shape for a procedure to be performed.

In one preferred and non-limiting aspect or embodiment, providing the pre-stressed membrane comprises: introducing a flowable elastomeric material to a mold; curing the elastomeric material within the mold; removing the cured membrane from the mold in an unstressed state; and stretching the unstressed membrane with a retaining frame to form the pre-stressed membrane.

In one preferred and non-limiting aspect or embodiment, contracting the membrane comprises threading sutures through corresponding anchoring holes of the membrane and through the dermal tissue to close the aperture of the membrane and incision.

In one preferred and non-limiting aspect or embodiment, provided is a kit of parts for adhered contractional wound closure comprising: an unstressed elastomeric membrane that can be pre-stressed by application of a stressing force, the membrane comprising: a body shaped for facilitating adhered contraction of a wound, one or more substantially annular collars extending from a surface of the body configured to form at least one receiving pocket, and a slit extending through at least a portion of the body; at least one retaining frame configured to be received within the receiving pocket formed by the one or more annular collars, the retaining frame being configured to apply the stressing force for pre-stressing the membrane, thereby causing the slit to expand to form an aperture; and an adhesive configured to attach the pre-stressed elastomeric membrane to dermal tissue of a patient at a surgical procedure site.

In one preferred and non-limiting aspect or embodiment, the elastomeric membrane comprises a plurality of tabs extending radially from the body of the membrane for varying a contracting force provided by the pre-stressed membrane when the membrane is adhered to the dermal tissue of the patient.

In one preferred and non-limiting aspect or embodiment, the at least one substantially annular collar comprises one or more radially extending through holes positioned for anchoring sutures for closing an incision.

In one preferred and non-limiting aspect or embodiment, the at least one substantially annular collar comprises an inner retaining collar comprising an outwardly directed flange and an outer retaining collar comprising an inwardly directed flange. In another preferred and non-limiting aspect or embodiment, the retaining frame comprises a first retaining ring configured to be received within the inner retaining collar and an outer ring configured to be received within the outer retaining collar. In a further preferred and non-limiting aspect or embodiment, the retaining frame comprises an annular shell comprising a disc shaped body configured to be received between the outwardly directed flange and the inwardly directed flange.

In one preferred and non-limiting aspect or embodiment, the at least one retaining frame comprises a plurality of retaining frames of different sizes and shapes, thereby permitting a user to select at least one of the plurality of retaining frames for use for a surgical procedure to be performed.

In one preferred and non-limiting aspect or embodiment, the elastomeric membrane further comprises guide lines embedded in or printed on a surface of the membrane, positioned to assist in visualization of stretching of the membrane and in alignment of an incision.

In one preferred and non-limiting aspect or embodiment, the elastomeric membrane comprises a silicone sheet having a thickness of about 0.5 mm to about 3.0 mm, and having a shore hardness of between shore 15 and 50 A.

In one preferred and non-limiting aspect or embodiment, the kit further comprises at least one adhesive bandage for securing the elastomeric membrane to the dermal tissue of the patient and/or for closing the aperture of the membrane and an incision formed at the surgical procedure site.

Further preferred and non-limiting aspects or embodiments of the present invention will now be described in the following numbered clauses:

Clause 1: A method for surgical wound closure including providing a pre-stressed elastomeric membrane comprising an aperture enclosed by at least a portion of the membrane; adhering the pre-stressed membrane to dermal tissue of a patient at a surgical procedure site; creating an incision through dermal tissue of a patient at the surgical procedure site at a location defined at least in part by the aperture of the membrane; and contracting the membrane and adhered dermal tissue, such that the aperture and incision created during the surgical procedure are at least partially closed.

Clause 2: The method of clause 1, wherein providing the pre-stressed membrane can comprise: providing an elastomeric membrane in an unstressed state; and retaining at least a portion of the membrane in a retaining frame, thereby stretching the membrane to a predetermined size and shape to form the pre-stressed membrane.

Clause 3: The method of clause 1 or 2, wherein contracting the membrane can comprise removing the retaining frame to release the membrane to apply a contracting force to the dermal tissue.

Clause 4: The method of any of clauses 1-3, wherein the elastomeric membrane can comprise at least one retaining collar extending from a surface of the membrane, and wherein retaining at least a portion of the membrane in a retaining frame can comprise mounting at least one retaining ring to the at least one retaining collar.

Clause 5: The method of any of clauses 1-4, wherein the elastomeric membrane can comprise at least an inner retaining collar and an outer retaining collar. The collars can extend from a surface of the membrane. Retaining at least a portion of the membrane in a retaining frame can comprise mounting at least one disc-shaped annular retaining shell between the inner retaining collar and the outer retaining collar.

Clause 6: The method of any of clauses 1-5, wherein the pre-stressed membrane can comprise a body and a plurality of tabs extending radially therefrom. The tabs can be configured to be adhered to dermal tissue of the patient at varying positions, thereby varying a contracting force provided to different portions of the dermal tissue by the membrane.

Clause 7: The method of any of clauses 1-6, wherein providing the pre-stressed elastomeric membrane comprises selecting a membrane of a suitable size and shape for a procedure to be performed from a plurality of different sized membranes.

Clause 8: The method of any of clauses 1-7, wherein providing the pre-stressed elastomeric membrane can comprise sizing the membrane by trimming portions of the membrane to produce an elastomeric membrane of an appropriate size and shape for a procedure to be performed.

Clause 9: The method of any of clauses 1-8, wherein providing the pre-stressed membrane can comprise: introducing a flowable elastomeric material to a mold; curing the elastomeric material within the mold; removing the cured membrane from the mold in an unstressed state; and stretching the unstressed membrane with a retaining frame to form the pre-stressed membrane.

Clause 10: The method of any of clauses 1-9, wherein contracting the membrane can comprise threading sutures through corresponding anchoring holes of the membrane and through the dermal tissue to close the aperture of the membrane and incision.

Clause 11: A kit of parts for adhered contractional wound closure includes an unstressed elastomeric membrane that can be pre-stressed by application of a stressing force. The membrane can include: a body shaped for facilitating adhered contraction of a wound, one or more substantially annular collars extending from a surface of the body configured to form at least one receiving pocket, and a slit extending through at least a portion of the body. The kit can further include at least one retaining frame configured to be received within the receiving pocket formed by the one or more annular collars and an adhesive configured to attach the pre-stressed elastomeric membrane to dermal tissue of a patient at a surgical procedure site. The retaining frame can be configured to apply the stressing force for pre-stressing the membrane, thereby causing the slit to expand to form an aperture.

Clause 12: The kit of clause 11, wherein the elastomeric membrane can comprise a plurality of tabs extending radially from the body of the membrane for varying a contracting force provided by the pre-stressed membrane when the membrane is adhered to the dermal tissue of the patient.

Clause 13: The kit of clause 11 or 12, wherein the at least one substantially annular collar can comprise one or more radially extending through holes positioned for anchoring sutures for closing an incision or wound.

Clause 14: The kit of any of clauses 11-13, wherein the at least one substantially annular collar can comprise an inner retaining collar comprising an outwardly directed flange and an outer retaining collar comprising an inwardly directed flange.

Clause 15: The kit of any of clauses 11-14, wherein the retaining frame can comprise a first retaining ring configured to be received within the inner retaining collar and an outer ring configured to be received within the outer retaining collar.

Clause 16: The kit of any of clauses 11-15, wherein the retaining frame can comprise an annular shell comprising a disc shaped body configured to be received between the outwardly directed flange and the inwardly directed flange.

Clause 17: The kit of any of clauses 11-16, wherein the at least one retaining frame can comprise a plurality of retaining frames of different sizes and shapes, thereby permitting a user to select at least one of the plurality of retaining frames for use for a surgical procedure to be performed.

Clause 18: The kit of any of clauses 11-17, wherein the elastomeric membrane can further comprise guide lines embedded in or printed on a surface of the membrane, positioned to assist in visualization of stretching of the membrane and in alignment of an incision.

Clause 19: The kit of any of clauses 11-18, wherein the elastomeric membrane can comprise a silicone sheet having a thickness of about 0.5 mm to about 3.0 mm, and having a shore hardness of between shore 15 and 50 A.

Clause 20: The kit of any of clauses 11-19, further comprising at least one adhesive bandage for securing the elastomeric membrane to the dermal tissue of the patient and/or for closing the aperture of the membrane and an incision formed at the surgical procedure site.

These and other features and characteristics of the present invention, as well as the methods of operation and functions of the related elements of structures and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and the claims, the singular form of “a”, “an”, and “the” include plural referents unless the context clearly dictates otherwise.

### BRIEF DESCRIPTION OF THE DRAWINGS

Some of the advantages and features of the preferred aspects or embodiments of the invention have been summarized herein above. These embodiments, along with other potential aspects or embodiments of the invention, will become apparent to those skilled in the art when referencing the following drawings in conjunction with the detailed descriptions as they relate to the figures.

FIG. 1 is a block cross-sectional view along the abdomen midline with a transverse incision, the location of which is indicated on FIG. 5A, and in which the left side of the cross-sectional view illustrates an application of an elastomeric wound closure according to an aspect of the present disclosure, and in which a right side of the cross-sectional view illustrates the healed wound;

FIG. 2 is a schematic drawing of an unstressed elastomeric membrane, which can be configured to form a contractional or pre-stressed membrane, according to an aspect of the disclosure;

FIG. 3 is a schematic drawing of a contractional or pre-stressed elastic membrane, in which the left side of the drawing illustrates the stretched base elastomeric membrane with a semi-rigid retention layer adhered, and the right side of the drawing shows the membrane with excess elastomeric material trimmed away and including incision guide lines following the aperture edge;

FIG. 4A is a schematic drawing that illustrates application of the membrane as indicated for a partial abdominoplasty, and in which the patient's right side demonstrates the contractional or pre-stressed elastomeric membrane adhered to the patient;

FIG. 4B is a corresponding cross-sectional view taken along line A-A′ of FIG. 4A;

FIG. 4C is a corresponding cross-sectional view taken along line B-B′ of FIG. 4A;

FIG. 5A is a schematic drawing that illustrates closure of a partial abdominoplasty wound, in which the patient's right side demonstrates the contractional or pre-stressed elastomeric membrane adhered to the patient and drawn together with sutures and adhesive bandages, to close and stabilize the initial incision;

FIG. 5B is a corresponding cross-sectional view taken along line C-C′ in FIG. 5A, in which the patient's left side illustrates a final healed wound;

FIG. 5C is a corresponding cross-sectional view taken along line D-D′ of FIG. 5A;

FIG. 6A is an un-stretched molded membrane according to another aspect of the present disclosure;

FIG. 6B is a cross-sectional view of the membrane of FIG. 6A taken at line E-E′ of FIG. 6A;

FIG. 7A is a front view of the membrane of FIG. 6A stretched and retained to a desired aperture size and shape, and in which the left side of the membrane illustrates use of two retaining rings to achieve the desired configuration, and in which the right side of the membrane illustrates use of a retaining shell to achieve the desired configuration;

FIG. 7B is a cross-sectional view taken along line F-F′ of FIG. 7A, and in which the membrane is retained by inner and outer retaining rings; and

FIG. 7C is a cross-section view taken along line G-G′ of FIG. 7A, and in which the membrane is retained by a retaining shell.

### DESCRIPTION OF THE INVENTION

For purposes of the description hereinafter, the terms “end”, “upper”, “lower”, “right”, “left”, “vertical”, “horizontal”, “top”, “bottom”, “lateral”, “longitudinal” and derivatives thereof shall relate to the invention as it is oriented in the drawing figures. However, it is to be understood that the invention may assume various alternative variations and step or stage sequences, except where expressly specified to the contrary. It is also to be understood that the specific devices and processes illustrated in the attached drawings, and described in the following specification, are simply exemplary embodiments or aspects of the invention. Hence, specific dimensions and other physical characteristics related to the embodiments or aspects disclosed herein are not to be considered as limiting.

The drawings generally show a preferred aspect or embodiment of the invention utilized for a partial abdominoplasty. While the descriptions of this invention relate to partial abdominoplasty, aspects or embodiments of this invention should not be interpreted in any way as limiting the invention to abdominoplasty. Furthermore, modifications, concepts, and applications of the invention are to be interpreted by those skilled in the art as being encompassed, but not limited to the illustrations and descriptions herein.

According to a preferred and non-limiting aspect or embodiment, a membrane with elastomeric properties that can be temporarily adhered to the epidermis of the skin is disclosed herein. The membrane can exhibit elasticity to enable sufficient forces to be transferred and retained by the membrane. The energy stored in the membrane will be released in a calculated and controlled manner to guide wound closure. The membrane can be provided as a surgical kit comprising at least one membrane, multiple retaining frames or structures of various sizes and shapes for stretching (e.g., pre-stressing) the membrane to a suitable size and shape for a surgical procedure to be performed, and adhesives for securing the membrane to dermal tissue of a patient. Adhesives can include pressure sensitive medical grade silicone adhesives or other flowable adhesive compositions, as well as adhesive bandages.

In some preferred and non-limiting aspects, embodiments, or examples, the membrane and wound closure method disclosed herein can be utilized with different types of abdominoplasty procedures. For purposes of illustration and description, use of the membrane for a partial abdominoplasty will be described herein. However, aspects of this invention are not to be construed as limited to abdominoplasty. Instead, the partial abdominoplasty procedure is provided to illustrate how surgeons can apply this invention to achieve favorable outcomes for a variety of surgical procedures. Abdominoplasty was chosen as exemplary because it represents a wound closure of significant size. Such procedures also demonstrate ability of this invention to assist with scarred and stretched tissue removal. These represent planned surgeries where achieving desired cosmetic outcomes are the motivation. Beyond the abdominoplasty example presented herein, this invention offers surgeons kits of wound closure devices that can be used in a variety of acute trauma situations. Rapid closure and stabilization of wounds can make the difference in saving lives. Such kits are compact, versatile, and can be valuable for emergency medics in the field, as well as in hospitals. This invention is intended to provide surgeons with another tool or means to facilitate the best possible wound closure along with improved cosmetic results. The elastomeric characteristics can be manipulated to direct contractional forces to assist wound closure and to stabilize tissues to enhance healing and reduce scarring.

Exemplary Elastomeric Membrane

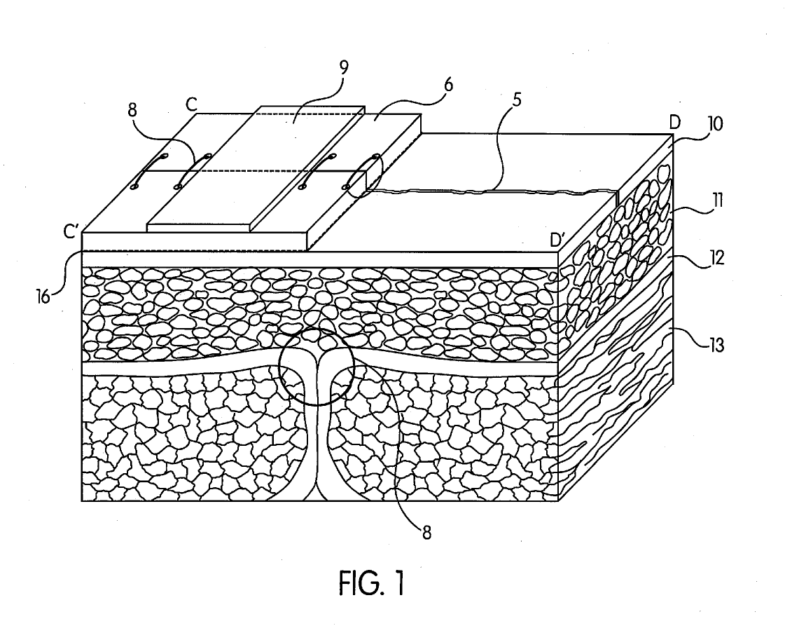

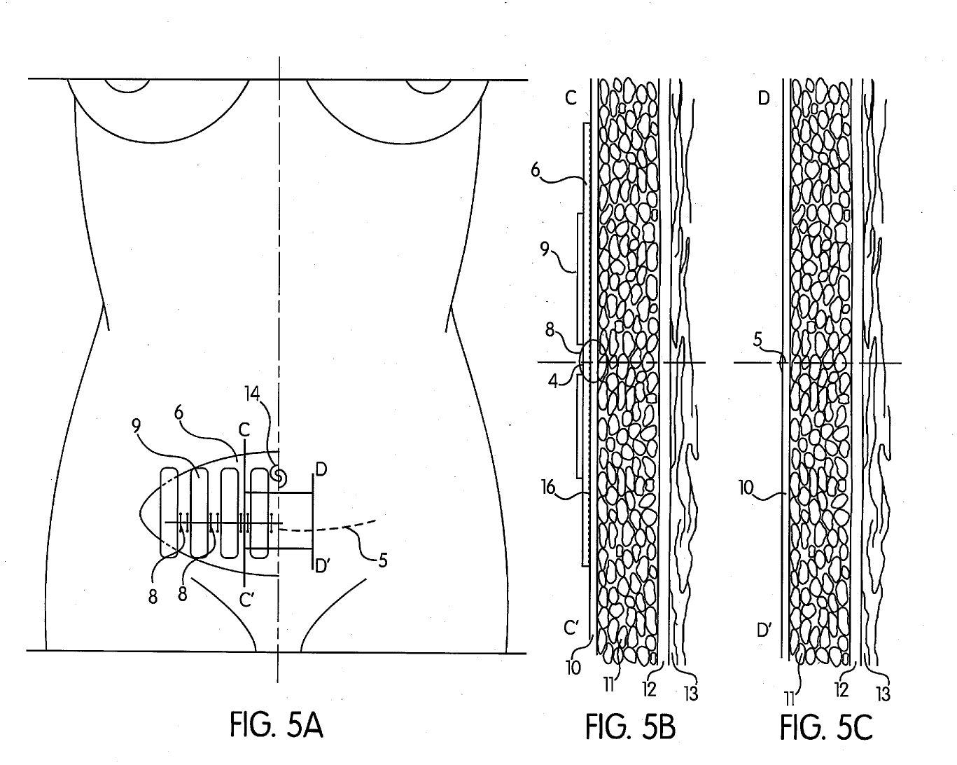

With reference to FIG. 1, a block cross-sectional view along the abdomen midline of a patient with a transverse incision is illustrated. The location of this view is indicated by lines C′-D′ and D-C (shown on FIG. 5A). In some preferred and non-limiting aspects, embodiments, or examples, the block is approximately 7×7 cm and approximately 5 cm deep. A skin layer 10 (shown in FIG. 1) is a simplified representation of the dermal layer. An elastomeric membrane 6 is adhered to the outer most epidermis of the skin layer 10. Under the skin layer 10 is adipose tissue or subcutaneous fat 11. The subcutaneous fat layer 11 is extremely variable in the general population. The preferred and non-limiting aspect, embodiment, or example illustrated in FIG. 1 is of an individual with minimal body fat. The sectional view includes a partial view of the rectus abdominis muscle 13 and anterior rectus abdominis fascia 12. The fascia 12 adjoins along the midline to divide the muscles 13. Surgical treatment during abdominoplasty can include the suturing together the muscles 13 and fascia 12 along the midline. The muscle repair is generally required, but variable depending on muscular laxity and tissue damage. A deep suture 8 has been utilized for this purpose. The membrane 6 is illustrated on the left side of FIG. 1. In this presentation, the membrane 6 is in a final and contracted state with the wound closed to permit healing. The elastomeric membrane 6 is held in the contracted state by sutures 8 and bandages 9 positioned to adjoin the two wound edges and stabilize the closure. The membrane 6 is adhered to the skin's epidermis (skin layer 10) by adhesive layer(s) 16. The right side of the illustration in FIG. 1 is representative of a healed wound with a small residual fine line scar 5, where the edges have healed together.

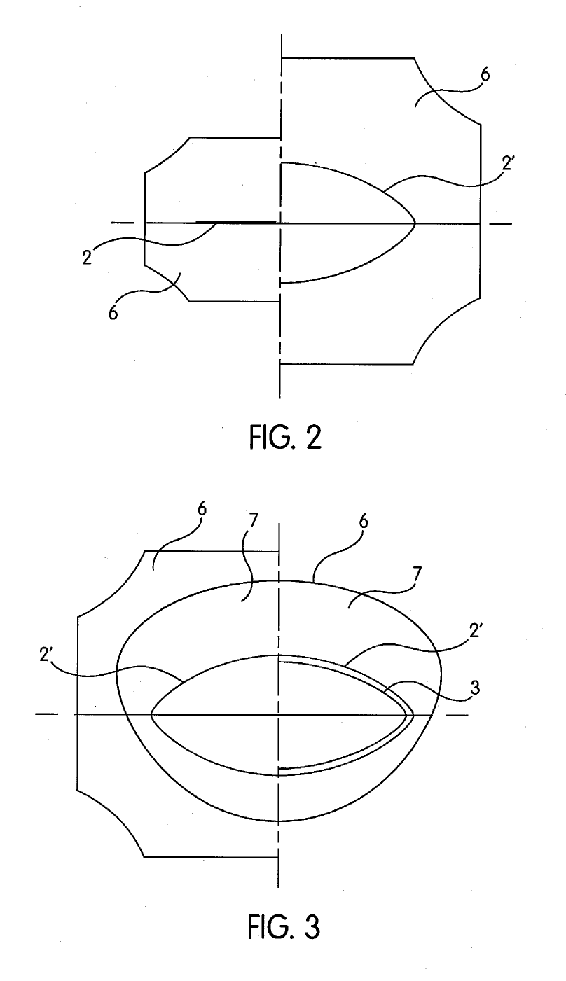

With reference to FIG. 2, the elastomeric membrane 6 is illustrated in an unstressed state (left side of FIG. 2) and in a stretched or pre-stressed state (right side of FIG. 2). The elastomeric membrane 6 is selected for the appropriate properties required. In one preferred and non-limiting aspect, embodiment, or example, the potential sizes of the membrane 6 in the unstressed state range from about 2 to about 30 centimeters across. On the left side of FIG. 2, the aperture line 2 is straight; however, in one preferred and non-limiting aspect, embodiment, or example, it can also be curved.

In some preferred and non-limiting aspects, embodiments, or examples, the elastomeric membrane 6 is formed from silicone. High performance silicone elastomers are widely used in the medical and prosthetic industry. Silicones are available with various properties. Elongation percentage and tear strength are of particular importance to this invention. Pre-formed silicone sheets or molded membranes with a hardness durometer ranging between about shore 15 to 50 A, preferably between about shore 20 to 35 A, and more preferably between about shore 25 to 30 A, would enable most applications. Effective thicknesses of the primary membranes structure will typically range from about 0.5 to about 3.0 mm. Some of the higher performance silicones can withstand elongation of 800% and exhibit significant tear strength. A variety of elastomeric materials can be utilized to achieve the desired properties, and such properties vary depending on the application needed.

In some preferred and non-limiting aspects, embodiments, or examples, oxygen transmission and general breathability characteristics can influence selection of membranes 6 for different applications. The membranes 6 can incorporate surface treatments to enhance healing. Anti-microbial treatments can also be utilized to enhance membrane attributes. Such treatments can be superficially applied or integral to the elastomeric membrane matrix. Despite variability among applications, there is a general continuity to the production and utilization of this invention.

In one preferred and non-limiting aspect, embodiment, or example, an adhesive, such as the adhesive layer 16 (shown in FIG. 1), can be applied prior to stretching the membrane 6. In some preferred and non-limiting aspects, embodiments, or examples, both sides of the membrane 6 are configured to contact the adhesive. One side of the membrane 6 is adhered to the skin layer 10 and the other side of the membrane 6 can be adhered to a retention layer 7 (shown in FIG. 3). Application of the adhesive or adhesive primer may be required or advantageous at this stage. Healing agents and or antibiotics can be applied at any stage of the formation process or previously integrated into the matrix of the elastomeric base material. On the right side of the drawing in FIG. 2, the membrane 6 is stretched as indicated by the increased area and scalloped corners. The stretching process can be varied to produce the desired aperture 2′. In general, the membrane 6 is stretched greater along the longitudinal axis compared to the transverse axis. The wound closure will require significantly more contraction perpendicularly to the transverse incision lines. The size of the stretched membrane can vary in the range of about 3 to about 50 centimeters across. The variability of elastomeric membrane materials is vast. Such variability contributes to the range of applications of this invention.

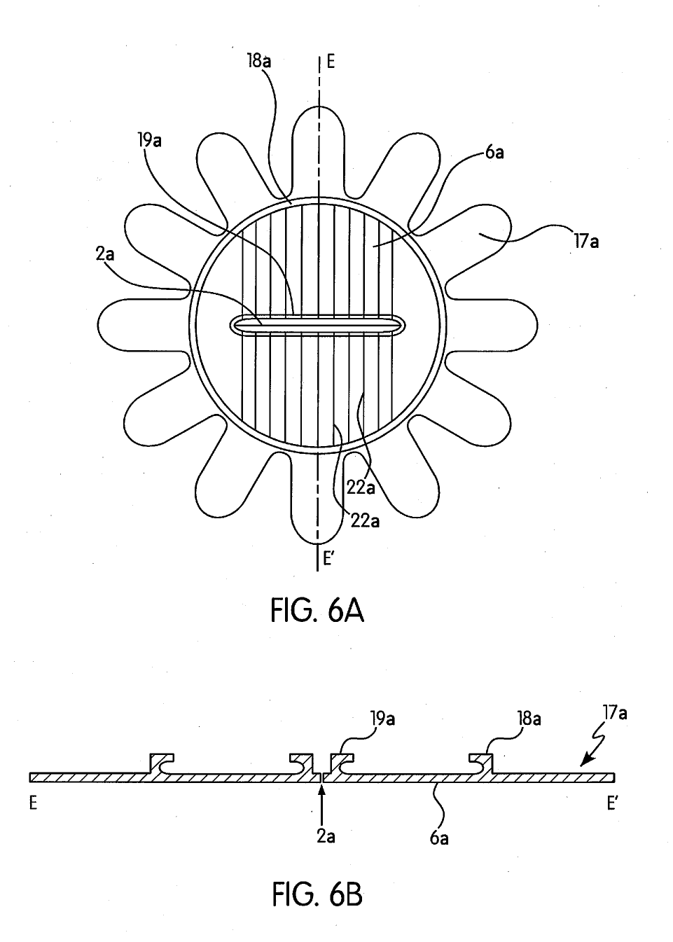

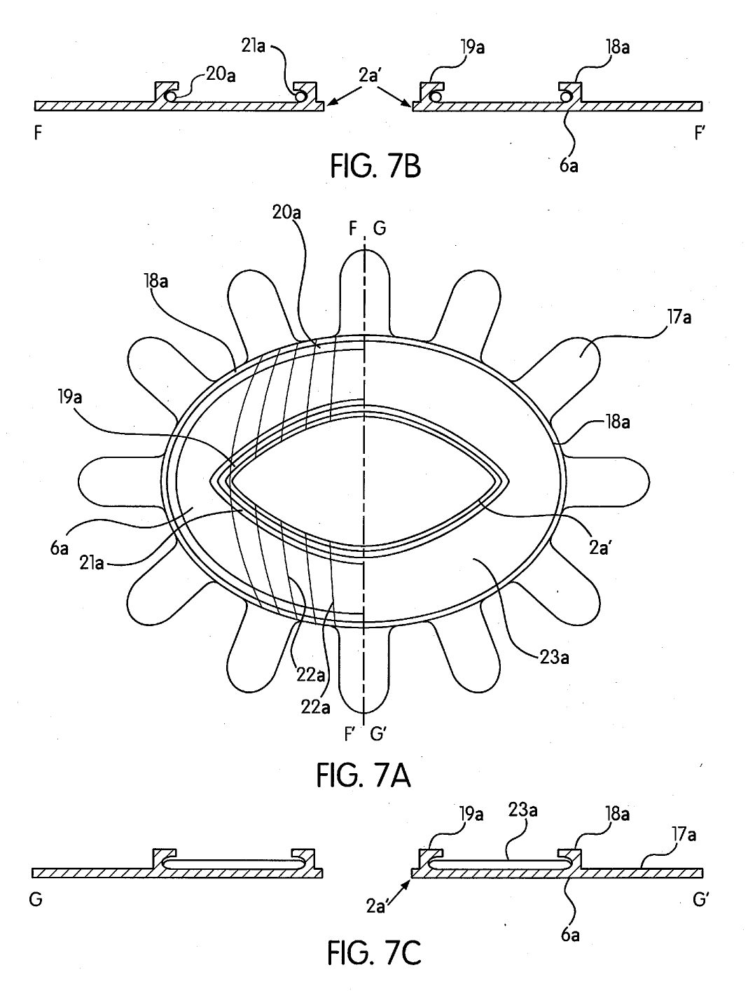

With reference to FIG. 3, another divided schematic drawing of the elastomeric membrane 6 according to one preferred and non-limiting aspect, embodiment, or example is illustrated. The left side of the drawing illustrates the stretched base elastomeric membrane 6 in a state producing the desired contractive forces and aperture 2′. A semi-rigid retention layer 7 is adhered to the membrane 6. The shape of the retention layer 7 is approximated for the application. The adhesive used must be strong enough to stabilize the membrane 6, yet still be able to delaminate when required. A laminating adhesive will vary depending on the materials used for the elastomeric membrane 6 and retention layer 7. In one preferred and non-limiting aspect, embodiment, or example, the adhesive can be applied to the membrane 6 prior to stretching and also after stretching as indicated. As described above, the adhesive can be applied to one surface or both surfaces of the membrane 6 depending on the properties of the materials involved. On the right side of the drawing in FIG. 3, excess elastomeric membrane 6 is trimmed to conform to the shape of the retention layer 7. The surgeon can still trim the membrane complex during the procedure to optimize the fit for application. One or more incision guide lines 3 are transferred or scribed on a surface of the membrane 6. In one preferred and non-limiting aspect, embodiment, or example, the incision guide line 3 can be a substantially circular line enclosed by the aperture 2′. If the surgeon is confident regarding the final incision, it can be mechanically inscribed with precision in advance. The surgeon then has a precise guide for the scalpel during surgery. In addition, various lines or grids can be indicated on the membrane 6 to assist the surgeon with alignment during wound closure. Exemplary guidelines 22a are shown in FIG. 6A and FIG. 7A. In some preferred and non-limiting aspects, embodiments, or examples, surgeons can customize and make modifications to the membrane 6 as they deem necessary during a procedure.

Exemplary Abdominoplasty Procedure

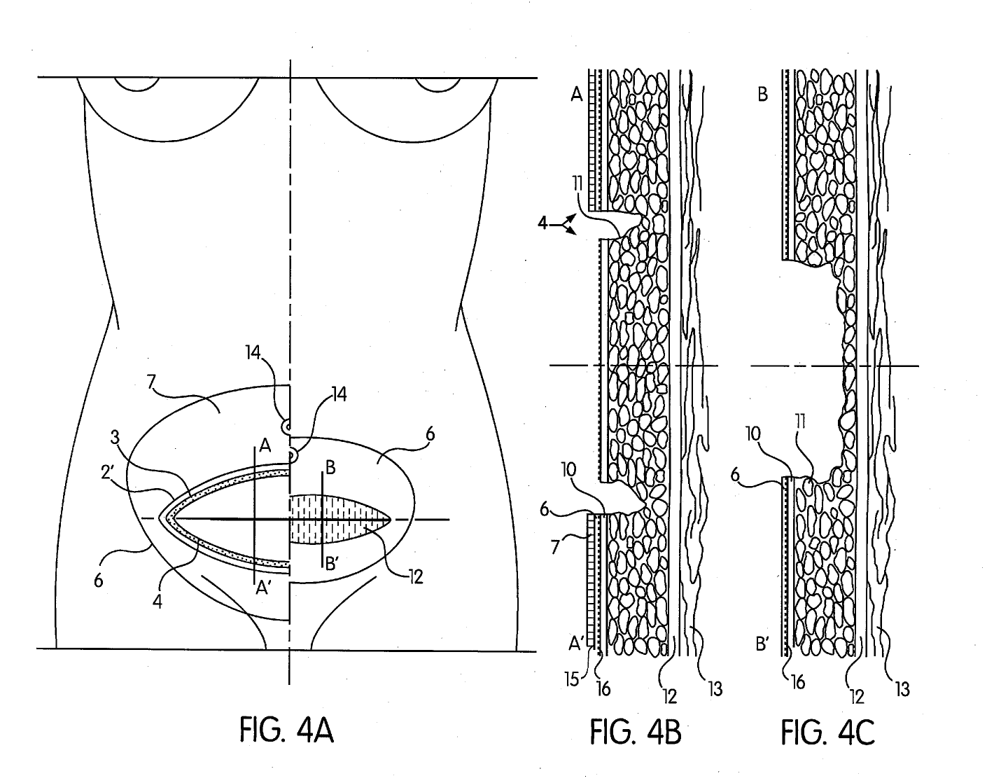

Having generally described the shape and function of certain preferred and non-limiting aspects, embodiments, or examples of the elastomeric membrane 6, use of the membrane 6 for wound closure for an abdominoplasty procedure will now be described in detail. With reference to FIG. 4A, a schematic drawing of the membrane 6 being used in the exemplary procedure according to one preferred and non-limiting aspect, embodiment, or example is illustrated. As was the case with previously described figures, the schematic drawing in FIG. 4A is divided. The patient's right side demonstrates the contractional elastomeric membrane 6 along with the laminated retention layer 7, as it would be adhered to the patient. In use, the surgeon fits the membrane and positions it according to a surgical plan or design. Once the membrane 6 is in place, an initial incision 4 is made using the incision guide 3 along the aperture 2′. Dissection beyond the initial incision 4 is easy to control with all the tissues being stabilized and delineated by the supporting membrane 6. The surgeon has freedom to manipulate the membrane 6 and the procedural order as deemed appropriate.

A corresponding cross-sectional view taken along line A-A′ (shown in FIG. 4A) is illustrated by FIG. 4B. The involved tissues are illustrated in this sectional view. An adhesive 15 between the membrane 6 and retention layer 7 and the adhesive layer 16 between the membrane 6 and epidermis (skin layer 10) are also indicated. The patient's left side of FIG. 4A has the tissue removed exposing the anterior rectus abdominis fascia 12. At this stage, the surgeon performs additional procedures and preparations as deemed necessary. The retention layer 7 is delaminated allowing the remaining elastomeric membrane 6 to contract rendering a greatly reduced wound size for closure. Movement of the tissues is evident by noting the displacement of the navel 14 with contraction. A corresponding cross-section view taken along line B-B′ is shown in FIG. 4C. The involved tissues (e.g., skin layer 10) are illustrated in this sectional view. The adhesive layer 16 between the membrane and epidermis (skin layer 10) is also illustrated.

With reference to FIG. 5A, a schematic drawing that illustrates closure of the partial abdominoplasty wound with the elastomeric membrane 6 according to one preferred and non-limiting aspect, embodiment, or example is illustrated. Many variations are possible ranging from pre-designed suture-less closures to basic membranes 6 allowing the surgeon to employ standard closure techniques as well as hybrids, of the two. As shown in FIG. 5A, the patient's right side demonstrates the contractional elastomeric membrane 6 adhered to the patient's abdomen and drawn together with sutures 8 and adhesive bandages 9. The initial incision has been closed and stabilized for wound healing. The surgeon may choose to trim sections of the membrane 6 along the wound closure margins. This will allow exposure of the underlying tissues if necessary for observation and management of healing.

A corresponding sectional view taken along line C-C′ is illustrated in FIG. 5B. The involved tissues are illustrated in this sectional view. The adhesive 16 between the membrane 6 and epidermis (skin layer 10) is illustrated along with a bandage 9 and suture 8. As shown in FIG. 5A, the patient's left side illustrates a final healed wound scar 5 after the membrane 6 has been removed. To remove the membrane 6, the surgeon determines an appropriate time for removal of the membrane 6 and then removes any sutures 8 to release the membrane 6. Medical solvents designed for removal of adhesive residue can also be utilized. With this preferred and non-limiting aspect, embodiment, or example, the healed incision line takes a slightly curved contour, and the resulting scar 5 is very minimal and smooth. A corresponding cross-sectional view of the healed tissue taken along line D-D′ is illustrated in FIG. 5C.

Exemplary Wound Closure Method

Having described the elastomeric membrane 6 both generally and in connection with an adominoplasty procedure, one preferred and non-limiting aspect, embodiment, or example of a method for preparing the membrane 6 and for using the prepared membrane 6 for a wound closure will be discussed in detail. Initially, a membrane 6 in an un-stretched or un-stressed state is provided. The membrane 6 can be formed from a single or multiple elastomeric layers and potentially multiple durometers. The resulting membrane 6 is stretched to desired quantifiable amounts. The stretching can be measured as a percentage of elongation or based on a required stretching force. Once the desired configuration is attained the membrane 6 is temporarily adhered to or retained in a semi-rigid shell. Another option for retaining the stretched membrane 6 is to use retaining rings or a hybrid approach of retaining techniques to adhere the membrane 6 to the shell. Thus, the desired elastic potential of the membrane can be retained and set for further preparation. The stabilized membrane 6 can potentially be cut or perforated for design needs or for enhancing breathability and fluid transmission where fluids may need to pass. In one preferred and non-limiting aspect, embodiment, or example, the membrane 6 requires structures to adhere to the targeted dermal tissues where the incision outline is delineated. There are many medical and prosthetic adhesives available with proven ability to temporarily adhere elastomeric materials to dermal tissues. Silicone adhesives dispersed in pentafluoropropane are a suitable preferred and non-limiting aspect, embodiment, or example.

In some preferred and non-limiting aspects, embodiments, or examples, the dermal tissue will need to have hair removed and prepared with adhesive primer, or its own adhesive layer, prior to attaching the membrane to the dermal tissue. Once the membrane 6 is in place, an incision can be performed through an incision aperture 2′ or opening. The incision aperture 2′ designed on the membrane provides guidance to create a controlled clean incision. The advantage of such an aperture 2′ will be apparent upon wound closure. Specifically, the aperture 2′ allows for removal of more tissue while still having a viable closure in cases where infected or necrotic tissue is being removed. Accordingly, the surgeon has greater wound closure potential by choice or necessity in various applications. For example, the surgeon can manually manipulate the aperture 2′ size and shape to design the incision lines 3 on the membrane 6 or to utilize a predetermined pattern. In one preferred and non-limiting aspect, embodiment, or example, the incision lines 3 can be cut, printed, or molded into the membrane and then utilized to guide the scalpel when the incision is performed. In some preferred and non-limiting aspects, embodiments, or examples, computer assisted design and mechanical cutting of the guide lines can be performed. Further, in some preferred and non-limiting aspects, embodiments, or examples, the ability to design incisions provides increased flexibility in performance of surgical procedures.

With planned surgeries, the membrane 6 can be available from a set series or custom ordered as needed. Surgeons can also design, manipulate and trim a membrane to their needs. In some preferred and non-limiting aspects, embodiments, or examples, the membrane 6 along with retaining and adhering structures, described herein, can be provided as a kit comprising one or more membranes 6 that can be used for a variety of wound closure needs. In the case of partial abdominoplasties, many surgeries will be serviced by pre-made designs. Abdominoplasties are one good example of how a planned surgery can proceed. In one preferred and non-limiting aspect, embodiment, or example, at a pre-surgical consultation a membrane 6 is selected based on measurements. Surgery proceeds with preparation of the abdominal area removing hair, cleaning, marking guide lines and application of adhesive. The selected membrane is applied according to the surgeons plan. The surgeon then uses an incision aperture guide, such as the incision lines 3 discussed herein, to remove the tissue leaving the wound with precise edges. Once the retaining layer 7 or rings are removed, the adhered elastomeric layer(s) of the membrane contract in a predictable manner. With the tissue removed, the abdominal tissue will lay flat and be supported by the contracted adhered membrane. The surgeon can then perform any additional procedures and prepare for wound closure.

Wound closure can be facilitated by a variety of methods. In some preferred and non-limiting aspects, embodiments, or examples, suturing techniques can be utilized in conjunction with membrane backed dermal tissues. The membrane 6 provides support and stability to the involved tissues. An advantage of such membrane backed dermal tissues is the ability to utilize the membrane 6 for the final superficial closure. The two wound edges have equal or near equal linear lengths and can have corresponding reference marks to align the opposing edges. Internal sutures can be placed by the surgeon as indicated for wound strength. The final closure can be achieved with external adhesive strips 9 or other external means. Flanges molded into the membrane 6, which can encircle the wound, can be used by the surgeon to aid with tensioning and distribution of wound closing forces. The adhered membrane 6 can also accommodate the attachment of anchoring structures that can be glued to the membrane where indicated. In some preferred and non-limiting aspects, embodiments, or examples, the flanges and anchoring points can also assist with securing wound dressings. The membrane 6 is configured to remain adhered to the skin tissue of the patient until sufficient healing and wound strength are attained. When removed, the wound will have a clean suture-less scar without apparent stress lines.

Exemplary Membrane and Retaining Frame

According to one preferred and non-limiting aspect, embodiment, or example, an elastomeric membrane 6a and retaining frame includes one or more retaining rings 20a, 21a and/or a retaining shell 23a, as illustrated in FIGS. 6A-7C. With reference to FIG. 6A, the membrane 6a is illustrated in an un-stretched or un-stressed state. The membrane 6a includes molded features to enhance the surgeon's ability to customize and prepare the membrane 6a. In some preferred and non-limiting aspects, embodiments, or examples, the membrane 6a and its features are formed by cast molding or injection molding. As shown in FIG. 6A, the membrane 6a can comprise an inner retaining collar 19a and an outer retaining collar 18a. The collars 18a, 19a are configured to create pockets suitable for receiving retaining devices, such as a retaining frame, to stretch the membrane 6a. While the membrane 6 in FIG. 6A includes two retaining collars 18a, 19a, membranes including only an outer retaining collar 18a can also be used. The collars 18a, 19a can have a variety of through holes or anchoring holes that the surgeon can use as attachments to assist with techniques for suturing the wound. In some preferred and non-limiting aspects, embodiments, or examples, the collars 18a, 19a can be cut off once they have served their purpose of retaining the membrane 6a during preparation, and prior to adhering the membrane to the wound site. The membrane 6a can also comprise guide lines 22a or grids that help visualize the stretching and assist with alignment of the incision margins. The membrane 6a can also include tabs 17a. The tabs 17a are configured to enhance adhesion to the skin and can be used to direct and vary forces of contraction. The tabs 17a are molded to a set pattern in this preferred and non-limiting aspect, embodiment, or example. For example, the membrane 6a can comprise about 6 to about 20 tabs extending radially from a body of the membrane 6a in a substantially symmetrical arrangement. The tabs 17a can be trimmed and/or entirely cut off if required. A continuous flange or extension of the membrane 6a around the perimeter is another option. Such a flange creates a pocket when the membrane 6a is stretched. The surgeon can cut the flange during the procedure to create tabs 17a for adhesion where needed. Excess material can then be cut off. In the unstressed state, as shown in FIG. 6A, an aperture edge 2a of the membrane 6 can be very narrow (e.g., a slit extending through a portion of the membrane 6). However, in certain implementations, a wider aperture edge can be favorable. In such a configuration, the inner retaining collar 19a can be set back from the aperture edge 2a. Setting back the edge 2a, leaves more material for the surgeon to modify and trim during a procedure.

With reference to FIG. 7A, a schematic drawing of the membrane 6a in a stretched state according to one preferred and non-limiting aspect, embodiment, or example is illustrated. In the stretched state, the aperture edge 2a′ is expanded and retained. Guide lines 22a are bent reflecting the stretching of the membrane 6a that creates the aperture 2a′. FIG. 7A is a divided illustration, which illustrates two embodiments for retaining the membrane 6a. The left side of the schematic drawing of FIG. 7A illustrates use of an inner retaining ring 21a and an outer retaining ring 20a. The rings 20a, 21a fit into the pockets created by the respective inner retaining collar 19a and outer retaining collar 18a. The rings 20a, 21a can be malleable. A shape and/or size of the rings 20a, 21a can be adjusted to control the aperture 2a′ size and also to manipulate the amount of stretching and, therefore, the contraction potential of the membrane 6a.

In some preferred and non-limiting aspects, embodiments, or examples, the retaining rings 21a, 20a can also be shaped and sized to contour to the body shape of the wound area. The right side of the membrane 6a (shown in FIG. 7A) illustrates the same shape as the left side, but with a different retaining mechanism. Specifically, the preferred and non-limiting aspect, embodiment, or example illustrated on the right side of FIG. 7A uses a retaining shell 23a to set the desired shape and amount of contraction. The retaining shell 23a can be a substantially flat, annular disc formed from a suitable bendable but rigid material such as plastic or medical grade fiberboard. The surgeon can select from a number of standard sized retaining shells 23a or can customize, cut, or trim a shell 23a specifically to his or her needs. In FIG. 7B, a cross section of the membrane 6a taken along line F-F′ (shown in FIG. 7A) is illustrated. The inner retaining ring 21a and the outer retaining ring 20a are positioned in the pockets of the inner 19a retaining collar and the outer retaining collar 18a. The aperture edges 2a′ demark the potential incision width through this section. In FIG. 7C, a cross section taken along line G-G′ (shown in FIG. 7A) is illustrated. This configuration utilizes a retaining shell 23a. The retaining shell 23a fits in the pockets of the inner retaining collar 19a and the outer retaining collar 20a. Surgical wound closure kits can be assembled from these exemplary components.

For purposes of summarizing the invention, certain aspects, features and advantages of the invention have been described. It is herein to be understood that not all advantages of this invention can be achieved in relation to any particular embodiment. As such, the invention can be embodied in configurations to optimize one or various advantages. Surgical applications of the invention can be indicated for any one advantage or combination of advantages as indicated for treatment.

While several embodiments of the membrane, kit of parts for wound closure, and method of adhering the membrane to a wound are shown in the accompanying figures and described hereinabove in detail, other embodiments will be apparent to, and readily made by, those skilled in the art without departing from the scope and spirit of the invention. For example, it is to be understood that this disclosure contemplates that, to the extent possible, one or more features of any embodiment can be combined with one or more features of any other embodiment. Accordingly, the foregoing description is intended to be illustrative rather than restrictive.

The invention claimed is:

1. A method for surgical wound closure comprising:
providing a pre-stressed elastomeric membrane comprising an aperture enclosed by at least a portion of the membrane;
adhering the pre-stressed membrane to dermal tissue of a patient at a surgical procedure site;
creating an incision through dermal tissue of a patient at the surgical procedure site at a location defined at least in part by the aperture of the membrane; and
contracting the membrane and adhered dermal tissue, such that the aperture and incision created during the surgical procedure are at least partially closed.

2. The method of claim 1, wherein providing the pre-stressed membrane comprises:
providing an elastomeric membrane in an unstressed state; and
retaining at least a portion of the membrane in a retaining frame, thereby stretching the membrane to a predetermined size and shape to form the pre-stressed membrane.

3. The method of claim 2, wherein contracting the membrane comprises removing the retaining frame to release the membrane to apply a contracting force to the dermal tissue.

4. The method of claim 2, wherein the elastomeric membrane comprises at least one retaining collar extending from a surface of the membrane, and wherein retaining at least a portion of the membrane in a retaining frame comprises mounting at least one retaining ring to the at least one retaining collar.

5. The method of claim 2, wherein the elastomeric membrane comprises at least an inner retaining collar and an outer retaining collar, the collars extending from a surface of the membrane, and wherein retaining at least a portion of the membrane in a retaining frame comprises mounting at least one disc-shaped annular retaining shell between the inner retaining collar and the outer retaining collar.

6. The method of claim 1, wherein the pre-stressed membrane comprises a body and a plurality of tabs extending radially therefrom, the tabs being configured to be adhered to dermal tissue of the patient at varying positions, thereby varying a contracting force provided to different portions of the dermal tissue by the membrane.

7. The method of claim 1, wherein providing the pre-stressed elastomeric membrane comprises selecting a membrane of a suitable size and shape for a procedure to be performed from a plurality of different sized membranes.

8. The method of claim 1, wherein providing the pre-stressed elastomeric membrane comprises sizing the membrane by trimming portions of the membrane to produce an elastomeric membrane of an appropriate size and shape for a procedure to be performed.

9. The method of claim 1, wherein providing the pre-stressed membrane comprises:
introducing a flowable elastomeric material to a mold;
curing the elastomeric material within the mold;
removing the cured membrane from the mold in an unstressed state; and
stretching the unstressed membrane with a retaining frame to form the pre-stressed membrane.

10. The method of claim 1, wherein contracting the membrane comprises threading sutures through corresponding anchoring holes of the membrane and through the dermal tissue to close the aperture of the membrane and incision.

11. A kit of parts for adhered contractional wound closure comprising:
an unstressed elastomeric membrane that can be pre-stressed by application of a stressing force, the membrane comprising:
a body shaped for facilitating adhered contraction of a wound,
one or more substantially annular collars extending from a surface of the body configured to form at least one receiving pocket, and
a slit extending through at least a portion of the body;
at least one retaining frame configured to be received within the receiving pocket formed by the one or more annular collars, the retaining frame being configured to apply the stressing force for pre-stressing the membrane, thereby causing the slit to expand to form an aperture; and
an adhesive configured to attach the pre-stressed elastomeric membrane to dermal tissue of a patient at a surgical procedure site.

12. The kit of claim 11, wherein the elastomeric membrane comprises a plurality of tabs extending radially from the body of the membrane for varying a contracting force provided by the pre-stressed membrane when the membrane is adhered to the dermal tissue of the patient.

13. The kit of claim 11, wherein the at least one substantially annular collar comprises one or more radially extending through holes positioned for anchoring sutures for closing an incision.

14. The kit of claim 11, wherein the at least one substantially annular collar comprises an inner retaining collar comprising an outwardly directed flange and an outer retaining collar comprising an inwardly directed flange.

15. The kit of claim 14, wherein the retaining frame comprises a first retaining ring configured to be received within the inner retaining collar and an outer ring configured to be received within the outer retaining collar.

16. The kit of claim 14, wherein the retaining frame comprises an annular shell comprising a disc shaped body configured to be received between the outwardly directed flange and the inwardly directed flange.

17. The kit of claim 11, wherein the at least one retaining frame comprises a plurality of retaining frames of different sizes and shapes, thereby permitting a user to select at least one of the plurality of retaining frames for use for a surgical procedure to be performed.

18. The kit of claim 11, wherein the elastomeric membrane further comprises guide lines embedded in or printed on a surface of the membrane, positioned to assist in visualization of stretching of the membrane and in alignment of an incision.

19. The kit of claim 11, wherein the elastomeric membrane comprises a silicone sheet having a thickness of about 0.5 mm to about 3.0 mm, and having a shore hardness of between shore 15 and 50 A.

20. The kit of claim 11, further comprising at least one adhesive bandage for securing the elastomeric membrane to the dermal tissue of the patient and/or for closing the aperture of the membrane and an incision formed at the surgical procedure site.
